(19) United States
(12) Patent Application Publication
(10) Pub. No.: US 2003/0077336 A1
(43) Pub. Date: Apr. 24, 2003

(54) Anti-inflammatory complex containing flaxseed oil

(75) Inventor: Philip B. Maffetone, St. Augustine, FL (US)

Correspondence Address: STEPTOE & JOHNSON LLP, 1330 Connecticut Avenue, N.W., Washington, DC, 20036, US

(73) Assignee: MAF Group, LLC, Boonton, NJ

(21) Appl. No.: 10/288,460

(22) Filed: Nov. 6, 2002

Related U.S. Application Data
(63) Continuation of application No. 09/972,992, filed on Oct. 10, 2001, now abandoned.

(51) Int. Cl. A61K 35/78; A61K 33/06; A61K 33/32; A61K 31/51; A61K 31/375; A61K 31/355

(52) U.S. Cl. 424/682; 424/756; 424/754; 424/736; 424/776; 514/276; 514/458; 514/474; 424/768; 424/643; 514/356

(57) ABSTRACT

A nutritional supplement includes flaxseed oil, ginger, sesame, citrus, garlic, tumeric, vitamins and minerals. The nutritional supplement preferably includes about 400 to 800 mg of flaxseed oil, about 50 to 100 mg of ginger, about 50 to 100 mg of sesame, about 50 to 100 mg of citrus, about 50 to 100 of garlic, about 10 to 30 mg of tumeric, about 6 to 10 mg of Vitamin C, about 1 to 5 IU of Vitamin E, about 0.25 to 1 mg of Vitamin B6, about 0.25 to 1 mg of niacin, about 3 to 6 mg of magnesium, and about 0.25 to 1 mg of zinc. The nutritional supplement may be used in a method of treating inflammation by administering to a patient in need thereof an effective dose of the nutritional supplement.

### BACKGROUND OF THE INVENTION

1. Field of the Invention

The present invention relates to a nutritional supplement containing a variety of naturally occurring substances useful for treating inflammatory conditions.

2. Description of the Related Art

Flaxseed oil contains significant amounts of alpha linolenic acid (ALA), an essential fatty acid.

ALA is an omega-3 fatty acid. The body turns ALA to a limited extent into eicosapentaenoic acid (EPA), which requires a variety of vitamins and minerals. EPA and DHA are omega-3 fatty acids also found in fish oil.

Flaxseed oil has anti-inflammatory activity. This requires conversion of ALA to EPA, which depends on enzymes that require various vitamins and minerals.

Flaxseed oil has been used in connection with the following conditions: constipation; high cholesterol; benign prostatic hyperplasia (BPH); ulcerative colitis; and vaginitis.

There are a number of known flaxseed oil products used as a natural non-animal source of the omega-3 essential fatty acids. Each capsule typically provides: 1000 mg flaxseed oil. A typical recommended dosage is 1 to 6 capsules daily.

For those who wish to replace fish oil with flaxseed oil, it takes approximately 11 g of ALA to make 1 g of EPA, indicating that a much higher volume of flaxseed oil is necessary. Optimal conversion of ALA to EPA requires the following nutrients: Vitamin C, Vitamin E, Vitamin B6, Vitamin B3, magnesium, and zinc.

There is a need in the art for a new and improved nutritional supplement containing flaxseed oil. There is also a need in the art for a nutritional supplement containing flaxseed oil with improved anti-inflammatory properties.

An object of the present invention is to provide a new and improved nutritional supplement containing flaxseed oil. Another object of the present invention is to provide a nutritional supplement containing flaxseed oil with improved anti-inflammatory properties. These and other objects of the present invention will become more apparent from a consideration of the following description and claims.

### BRIEF SUMMARY OF THE INVENTION

The nutritional supplement of the present invention includes flaxseed oil, ginger, sesame, citrus, garlic and tumeric as anti-inflammatory ingredients. The nutritional supplement also contains nutrients necessary for the conversion of ALA to EPA, namely, one or more vitamins and minerals. The nutritional supplement preferably includes about 400 to 800 mg of flaxseed oil, about 50 to 100 mg of ginger, about 50 to 100 mg of sesame, about 50 to 100 mg of citrus, about 50 to 100 mg of garlic, and about 10 to 30 mg of tumeric. The nutritional supplement also preferably includes about 6 to 10 mg of Vitamin C, about 1 to 5 IU of Vitamin E, about 0.25 to 1 mg of Vitamin B6, about 0.25 to 1 mg of niacin, about 3 to 6 mg of magnesium and about 0.25 to 1 mg of zinc. The ginger is preferably ginger root, the sesame is preferably sesame seed oil, the citrus is preferably citrus skin oil, and the garlic is preferably garlic powder (odorless).

The nutritional supplement of the present invention more preferably includes:

| | | |
|---|---|---|
| Flaxseed oil | 450 to 550 | mg |
| Ginger | 70 to 80 | mg |
| Sesame | 70 to 80 | mg |
| Citrus | 70 to 80 | mg |
| Garlic | 70 to 80 | mg |
| Tumeric | 10 to 30 | mg |
| Vitamin C | 6 to 10 | mg |
| Vitamin E | 1 to 5 | IU |
| Vitamin B6 | 0.25 to 1 | mg |
| Niacin | 0.25 to 1 | mg |
| Magnesium | 3 to 6 | mg |
| Zinc | 0.25 to 1 | mg. |

The nutritional supplement of the present invention most preferably contains:

| | | |
|---|---|---|
| Organic flaxseed oil | 500 | mg |
| Organic ginger root | 75 | mg |
| Organic sesame seed oil | 75 | mg |
| Organic citrus skin oil | 75 | mg |
| Organic garlic powder (odorless) | 75 | mg |
| Organic tumeric | 20 | mg |
| Vitamin C | 50 | mg |
| Vitamin E | 1 | IU |
| Vitamin B6 | 0.5 | mg |
| Niacin | 0.5 | mg |
| Magnesium | 4 | mg |
| Zinc | 0.5 | mg |

The anti-inflammatory complex containing flaxseed oil is a natural remedy, especially for the relief of chronic inflammation. Chronic inflammation is often an asymptomatic condition that may be the first stage of a variety of chronic diseases, including cancer, heart disease, neurodegenerative disorders (e.g., Alzheimer), and others.

Accordingly, the present invention also relates to a method of treating inflammation which includes administering to a patient in need thereof a nutritional supplement including flaxseed oil, ginger, sesame, citrus, garlic and tumeric, as well as one or more nutrients for the conversion of ALA to EPA. The preferred mode of administration is by capsule or the like. The recommended dosage is 5 to 20 capsules per day.

While the preferred use of the present invention is for inflammatory conditions, it will be understood that the present invention may be used as a nutritional supplement for any of the conditions flaxseed oil, ginger, sesame, citrus, garlic, or tumeric are known to be used for in the art.

### DETAILED DESCRIPTION OF THE INVENTION

In accordance with the present invention, a nutritional supplement is provided which includes flaxseed oil, ginger, sesame, citrus, garlic and tumeric, as well as one or more nutrients for the conversion of ALA to EPA.

The nutritional supplement most preferably contains about 400 to 800 mg, more preferably about 450 to 550 mg, and most preferably about 500 mg of flaxseed oil. Flaxseeds contain ALA, some of which can be converted to EPA providing an anti-inflammatory effect. As such, flaxseed oil has been used to treat inflammatory related conditions such as arthritis, colitis, cancer, heart disease and others.

Ginger is a perennial plant that grows in the United States, India, China, Mexico, and several other countries. The dried rhizome (underground stem or root) of ginger contains approximately 1-4% volatile oils. These are the medically active constituents of ginger, and they are also responsible for ginger's characteristic odor and taste. The aromatic principles include zingiberene and bisabolene, while the pungent principles are known as gingerols and shogaols.

Ginger is a classic tonic for the digestive tract, may be helpful for motion sickness, has anti-fungal and anti-oxidant effects, and may protect the stomach from the damaging effect of non-steroidal anti-inflammatory drugs. Ginger also has anti-inflammatory properties and has been used for the treatment of certain cancers, cardiovascular disease, thrombosis, arteriolsclerosis, atherosclerosis, hyperlipoproteinemia, and other conditions. The nutritional supplement of the present invention preferably contains about 50 to 100 mg, more preferably about 70 to 80 mg, and most preferably about 75 mg of ginger.

Sesame seed oil is derived from a plant species called Sesamum indicum, which is an herbaceous annual belonging to the pedaliaceae family. Sesame seed oil, which contains the lignan sesamin, has been used for a variety of therapeutic purposes. The oil is also used in certain cosmetics and skin softening products.

Sesame oil, which is also referred to as benne, gingili, or teel oil, is made from the black seeds of Sesamum indicum. Sesame seed oil has been shown to have anti-inflammatory, anti-oxidant, and anti-hypertensive properties, and as such is believed to have several other important properties, including anti-cancer and antibacterial effects. The nutritional supplement of the present invention preferably contains about 50 to 100 mg, more preferably about 70 to 80 mg, and most preferably about 75 mg of sesame seed oil.

Citrus skin oil can be prepared from a variety of citrus fruits, preferably oranges. Citrus skin oil contains significant levels of monoterpenes, including limonene, which have anti-inflammatory and anti-oxidant effects, with studies supporting their anti-cancer effects especially with cancers of the breast, colon and skin. The nutritional supplement of the present invention preferably contains about 50 to 100 mg, more preferably about 70 to 80 mg, and most preferably about 75 mg of citrus skin oil.

Garlic is closely related to onion and chives. The sulfur compound allicin, produced by crushing or chewing fresh garlic, in turn produces other sulfur compounds: ajoene, allyl sulfides, and vinyldithiins. Research suggests that garlic powder may have anti-inflammatory actions, along with antimicrobial, antithrombotic, antitumor, hypolipidaemic, and hypoglycemic effects. The nutritional supplement of the present invention preferably contains about 50 to 100 mg, more preferably about 70 to 80 mg, and most preferably about 75 mg of garlic powder (odorless).

Tumeric is the common name for Curcuma domestica Loir or Curcuma longa L. Tumeric is a perennial herb. The rhizomes are robust, fleshy and branch at right angles forming a dense clump. The rhizomes are aromatic, orange-brown in color on the outside and bright orange in the inside. The tubers are elliptical in shape, fleshy, white in color and grow vertically downwards. The rhizomes are the plant parts used. Tumeric, which contains a substance called curcumin, has both anti-inflammatory and anti-oxidant effects. As such, it has been shown to have anti-cancer effects. The nutritional supplement of the present invention preferably contains about 10 to 30 mg, and most preferably about 20 mg of tumeric.

The nutritional supplement also contains nutrients necessary for the conversion of ALA to EPA, namely, one or more vitamins and minerals, preferably in their natural forms. These vitamins and minerals have other beneficial properties as well.

Acerola extract is a natural source of Vitamin C (about 50 mg of Acerola extract provides about 8 mg of Vitamin C). Its botanical name is Malpighia glabra and its family is Mapighiaceae. The fruit originates from Brazil. Acerola has a high content of Vitamin C. 100 g of Acerola pulp can have as much as 17 g of Vitamin C. Vitamin C acts as a free radical scavenger. The nutritional supplement of the present invention preferably contains about 6 to 10 mg, most preferably about 8 mg of Vitamin C.

Vitamin E (also called alpha tocopherol) is an antioxidant that protects cell membranes and other fat-soluble parts of the body. The nutritional supplement of the present invention preferably contains about 1 to 5 IU of Vitamin E, most preferably about 1 IU of Vitamin E. The Vitamin E is preferably from soy oil.

Vitamin B6 (in its two forms called pyridoxal-5'-phosphate and pyridoxamine 5-phosphate) is essential in the processing of amino acids, proteins and some hormones. Vitamin B6 is needed to make serotonin, melatonin, and dopamine, along with other important substances. The nutritional supplement of the present invention preferably contains about 0.25 to 1 mg, most preferably about 0.5 mg of Vitamin B6. The Vitamin B6 is preferably from nutritional yeast.

Niacin (also called Vitamin B3) exists in a nicotinic acid and nicotinamide form. The body uses Vitamin B3 for many metabolic functions. The nutritional supplement of the present invention preferably contains about 0.25 to 1 mg, most preferably about 0.5 mg of niacin. The niacin is preferably from nutritional yeast.

Magnesium is needed for bone, protein, and fatty acid formation, making new cells, activating B vitamins, relaxing muscles, clotting blood, and forming ATP. The nutritional supplement of the present invention preferably contains about 3 to 6 mg, most preferably about 4 mg of magnesium.

Zinc is a component of more than 300 enzymes needed to repair wounds, maintain fertility in adults and growth in children, synthesize protein, help cells reproduce, preserve vision, boost immunity, and protect against free radicals, among other functions. The nutritional supplement of the present invention preferably contains about 0.25 to 1 mg, most preferably about 0.5 mg of zinc.

The nutritional supplement is preferably provided as a capsule and includes a liquid or dry inner filling of active ingredients and an outer shell. In the preferred embodiment, the inner filling is a liquid mixture which is contained within a gelatin capsule, such as a “softgel” type capsule. The inner filling includes flaxseed oil, ginger, sesame, citrus, garlic and tumeric, as well as one or more vitamins and minerals.

The components of the inner filling are preferably solubilized in a liquid to form a liquid mixture. Suitable liquids include vegetable oils such as sesame seed oil. The inner filling can contain other components, including fillers, and components useful for adjusting the isotonic properties of the filling (such as glycerin or beeswax).

When the inner filling of the nutritional supplement of the present invention is a liquid, the outer shell is preferably composed of a gelatin as well as other optional components such as glycerin and a coloring agent (such as carob). Such a nutritional supplement can be obtained by filling the outer shell with the premixed inner filling.

The recommended dosage is 5 to 20 capsules per day.

While the foregoing invention has been described in some detail for purposes of clarity and understanding, it will be appreciated by one skilled in the art from a reading of this disclosure that various changes in form and detail can be made without departing from the true scope of the invention and appended claims.

I claim:

1. A nutritional supplement comprising flaxseed oil, ginger, sesame, citrus, garlic, tumeric, and a nutrient for converting alpha linolenic acid to eicosapentaenoic acid.

2. The nutritional supplement of claim 1, comprising about 400 to 800 mg of flaxseed oil, about 50 to 100 mg of ginger, about 50 to 100 mg of sesame, about 50 to 100 mg of citrus, about 50 to 100 mg of garlic, about 10 to 30 mg of tumeric, and one or more nutrients wherein the nutrients are about 6 to 10 mg of Vitamin C, about 1 to 5 IU of Vitamin E, about 0.25 to 1 mg of Vitamin B6, about 0.25 to 1 mg of niacin, about 3 to 6 mg of magnesium, and about 0.25 to 1 mg of zinc.

3. The nutritional supplement of claim 1, wherein the ginger is organic ginger root.

4. The nutritional supplement of claim 1, wherein the sesame is organic sesame seed oil.

5. The nutritional supplement of claim 1, wherein the citrus is organic citrus skin oil.

6. The nutritional supplement of claim 1, wherein the garlic is garlic powder.

7. The nutritional supplement of claim 1, wherein the tumeric is organic tumeric.

8. A method of treating inflammation, comprising administering to a patient in need thereof an effective amount of a nutritional supplement comprising flaxseed oil, ginger, sesame, citrus, garlic, tumeric, and a nutrient for converting alpha linolenic acid to eicosapentaenoic acid.

9. The method of claim 8, wherein the nutritional supplement comprises about 400 to 800 mg of flaxseed oil, about 50 to 100 mg of ginger, about 50 to 100 mg of sesame, about 50 to 100 mg of citrus, 50 to 100 mg of garlic, 10 to 30 mg of tumeric, and one or more nutrients wherein the nutrients are about 6 to 10 mg of Vitamin C, about 1 to 5 IU of Vitamin E, about 0.25 to 1 mg of Vitamin B6, about 0.25 to 1 mg of niacin, about 3 to 6 mg of magnesium, and about 0.25 to 1 mg of zinc.

10. The method of claim 8, wherein the ginger is organic ginger root.

11. The method of claim 8, wherein the sesame is organic sesame seed oil.

12. The method of claim 8, wherein the citrus is organic citrus skin oil.

13. The method of claim 8, wherein the garlic is garlic powder.

14. The method of claim 8, wherein the tumeric is organic tumeric.

15. A nutritional supplement comprising:

| | | |
|---|---|---|
| Flaxseed oil | 450 to 550 | mg |
| Ginger | 70 to 80 | mg |
| Sesame oil | 70 to 80 | mg |
| Citrus | 70 to 80 | mg |
| Garlic | 70 to 80 | mg |
| Tumeric | 10 to 30 | mg |
| Vitamin C | 6 to 10 | mg |
| Vitamin E | 1 to 5 | IU |
| Vitamin B6 | 0.25 to 1 | mg |
| Niacin | 0.25 to 1 | mg |
| Magnesium | 3 to 6 | mg |
| Zinc | 0.25 to 1 | mg. |

16. An organic nutritional supplement comprising:

| | | |
|---|---|---|
| Flaxseed oil | 500 | mg |
| Ginger root | 75 | mg |
| Sesame seed oil | 75 | mg |
| Citrus skin oil | 75 | mg |
| Garlic powder | 75 | mg |
| Tumeric | 20 | mg |
| Vitamin C | 8 | mg |
| Vitamin E | 1 | IU |
| Vitamin B6 | 0.5 | mg |
| Niacin | 0.5 | mg |
| Magnesium | 4 | mg |
| Zinc | 0.5 | mg. |

17. A method of treating inflammation, comprising administering to a patient in need thereof an effective amount of a nutritional supplement comprising:

| | | |
|---|---|---|
| Flaxseed oil | 450 to 500 | mg |
| Ginger | 70 to 80 | mg |
| Sesame | 70 to 80 | mg |
| Citrus | 70 to 80 | mg |
| Garlic | 70 to 80 | mg |
| Tumeric | 10 to 30 | mg |
| Vitamin C | 6 to 10 | mg |
| Vitamin E | 1 to 5 | IU |
| Vitamin B6 | 0.25 to 1 | mg |
| Niacin | 0.25 to 1 | mg |
| Magnesium | 3 to 6 | mg |
| Zinc | 0.25 to 1 | mg. |

18. A method for treating inflammation, comprising administering to a patient in need thereof an effective amount of an organic nutritional supplement comprising:

| | | |
|---|---|---|
| Flaxseed oil | 500 | mg |
| Ginger root | 75 | mg |
| Sesame seed oil | 75 | mg |
| Citrus skin oil | 75 | mg |
| Garlic powder | 75 | mg |
| Tumeric | 20 | mg |
| Vitamin C | 8 | mg |
| Vitamin E | 1 | IU |
| Vitamin B6 | 0.5 | mg |
| Niacin | 0.5 | mg |
| Magnesium | 4 | mg |
| Zinc | 0.5 | mg. |
